(19) United States
(12) Patent Application Publication
(10) Pub. No.: US 2012/0310171 A1
(43) Pub. Date: Dec. 6, 2012

(54) Safety Device Mounting Arrangement

(75) Inventor: Barry Peter Liversidge, Langham Colchester (GB)

(73) Assignee: Barry Peter LIVERSIDGE, Langham, Colchester, Essex (GB)

(21) Appl. No.: 13/576,346

(22) PCT Filed: Feb. 1, 2011
(86) PCT No.: PCT/GB2011/050161

§ 371 Date: Aug. 23, 2012

(30) Foreign Application Priority Data
Feb. 1, 2010 (GB) 1001506.3
Oct. 18, 2010 (GB) 1017491.0

(51) Int. Cl. A61M 5/32

(52) U.S. Cl. 604/198

(57) ABSTRACT

A mount for a safety device for use in association with a medical needle projecting from a generally cylindrical carrier having three or four circumferentially-spaced ribs upstanding from and extending substantially parallel to the axis of the carrier. The mount has a tubular support including a wall having a central aperture through which the carrier extends when in use with the wall lying in a substantially radial plane with respect to the carrier. The aperture is profiled to surround the carrier and has recesses for receiving the carrier ribs. Portions of the wall defining the aperture and recesses engage the ribs of the carrier, with an interference fit between the ribs of the carrier and adjacent wall parts.

This invention relates to a mount for use in association with a medical needle projecting from a carrier, to facilitate the mounting thereon of a safety device adapted to confer protection to the needle. The invention extends to the combination of such a mount and a medical needle projecting from a carrier.

Modern medical practice frequently requires the use of a safety device to confer protection on a needle used for penetrating a human or animal body. The safety device may be passive, and so which operates fully automatically without requiring any action by a user of the needle, or active, which requires an action by the user. Either way, the safety device will be manufactured separately from the needle and then must be associated with the needle so as to confer protection thereto.

Some medical needles are mounted on a hub which is then packaged separately from a syringe or other medical injector with which the needle is to be used. When required for use, the needle is removed from its package and is fitted to the syringe, typically by way of a Luer-Slip or Luer-Lok taper connection. Other medical needles are permanently attached to a syringe in the course of the manufacture thereof, either by being staked-in with an adhesive or perhaps co-moulded with a plastic syringe. If protection is to be given to the needle, the safety device must then be mounted either on the needle hub, in the case of a separate needle, or on the syringe in the case of a syringe having a needle permanently attached thereto or where a separate needle has been fitted to the syringe.

There have been various proposals for the secure mounting of a safety device to a medical needle hub or to a syringe carrying a needle. Some of these require extensive modification to the needle hub or syringe and so can be used exclusively with a needle hub or syringe specifically adapted for use with the safety device. Others require the use of a special mount allowing the safety device to be connected to the syringe. Such arrangements normally require complex moulding of the needle hub or the syringe, frequently necessitating the use of moulds with side actions; in turn this significantly increases the manufacturing costs.

It is a principal aim of this invention to provide a mount for a safety device for use with a medical needle either provided with a mounting hub for securing to a syringe, or provided as an integral part of a syringe, and which is easy to implement and use.

According to one aspect of this invention, there is provided a mount for a safety device for use in association with a medical needle projecting from a generally cylindrical carrier including at least two circumferentially-spaced ribs upstanding from and extending substantially parallel to the axis of the carrier, said mount including a wall having a central aperture through which the carrier extends when in use with the wall lying in a substantially radial plane with respect to the carrier, the aperture being profiled so that portions of the wall defining the aperture engage with an interference fit the ribs of the carrier.

On fitting together the safety device and the carrier for the needle, the ribs of the carrier interengage with portions of the wall of the mount. By forming an interference fit between the ribs and the wall portions, the mount may be securely held on the carrier so securing the safety device on the carrier, surrounding the needle in order to confer protection thereto.

In a preferred embodiment, the aperture of the mount wall has a generally circular central region adapted to accommodate the generally cylindrical carrier. Further, the wall has a plurality of recesses extending generally radially away from the central region, each recess being adapted to receive a respective rib upstanding from the carrier.

Each recess of the mount wall may be profiled in order to provide a pair of opposed corners at the junction between the recess and the central aperture, the spacing between the corners being less than the width of a rib on the carrier with which the mount is to co-operate. In this way, the interference fit is created between said corners of the recess and the rib of the carrier which is received in the recess. In the alternative, or perhaps in addition, each recess has a radially outer surface defined by the wall and in use, the outer surface of the recess engages and forms an interference fit with the radially outer surface of the rib received in that recess.

In order to ensure that a sufficient interference fit is formed between the mount and a carrier, the mount may be made of a relatively hard material with respect to the material of the ribs of a carrier with which the mount is used. Also, the materials of the wall of the mount and the ribs may flex to some extent on fitting the mount to the carrier, to enhance the connection between the mount and the carrier.

The mount may be associated with a safety device so that when the mount is used with a carrier supporting a medical needle, protection is conferred on that needle. Such a safety device may have a sleeve slidable between shielding and non-shielding positions, the sleeve being slidably carried on the outer surface of the mount. The sleeve may be spring-urged to a shielding position and may have a mechanism arranged to lock or block the sleeve in its shielding position, after use of the device to perform a medical procedure.

According to a further aspect of this invention, there is provided a mount of this invention in combination with a medical needle projecting from a generally cylindrical carrier including at least two circumferentially-spaced ribs upstanding from and extending parallel to the axis of the carrier, with parts of the wall of the mount forming an interference fit with the ribs of the carrier thereby to hold the mount to the carrier.

The carrier may have two ribs, in which case those ribs should be substantially diametrically opposed. In the alternative, the carrier may have more than two ribs which should be substantially equi-spaced around the carrier. For example, there may be three ribs disposed at 120° to each other or four ribs arranged mutually at right angles, though it would be possible to have other numbers of ribs. The wall of the mount should be similarly profiled, for receiving the ribs of the carrier.

Other features of this invention are defined in the subsidiary claims appended hereto and to which reference should be made.

By way of example only, this invention will now be described in more detail and specific embodiments thereof given, referring to the accompanying drawings in which:

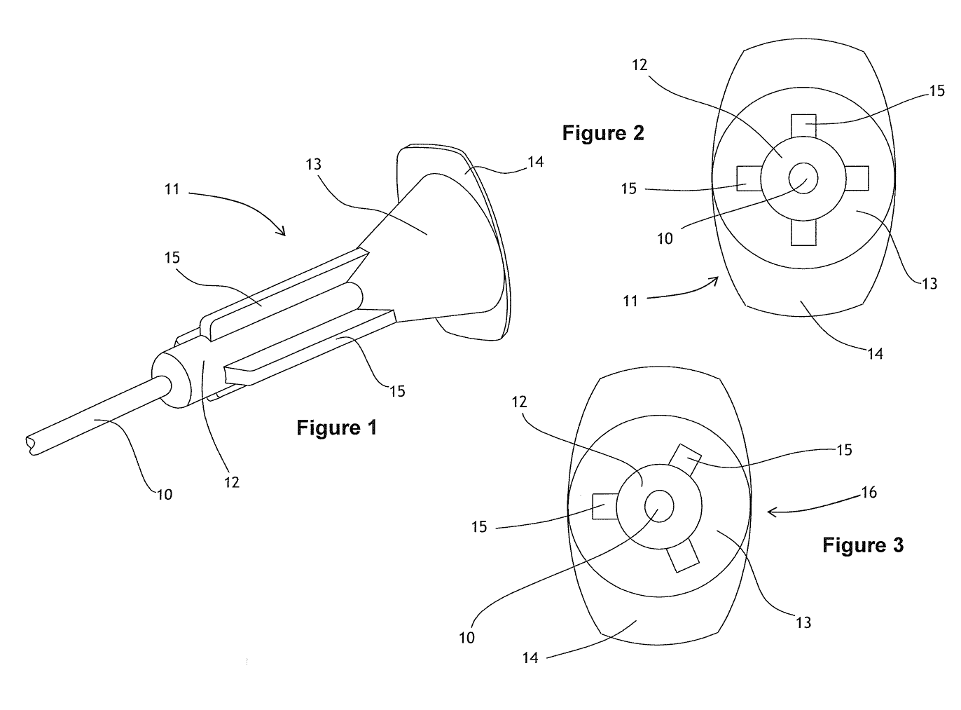

FIG. 1 is an isometric view of a conventional needle and hub, as widely used throughout the medical industry;

FIG. 2 is an end view on the needle and hub of FIG. 1;

FIG. 3 is an end view on an alternative needle and hub;

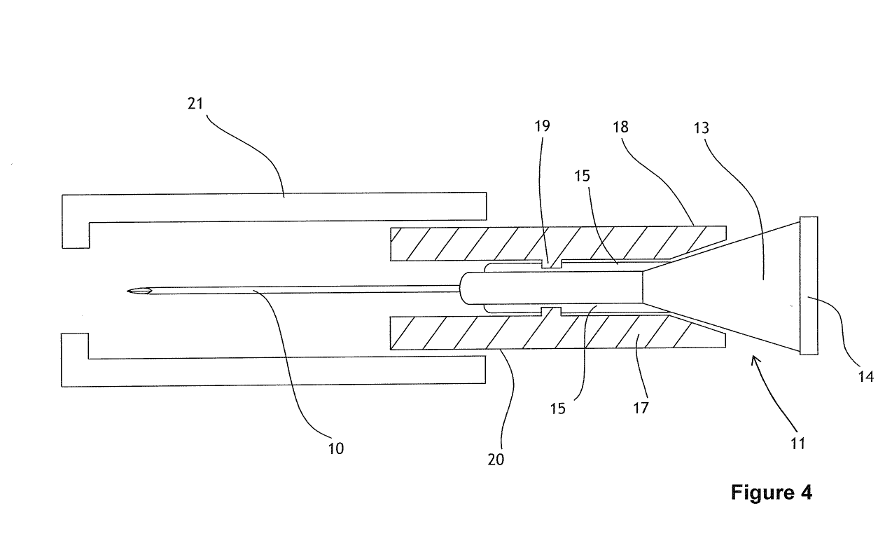

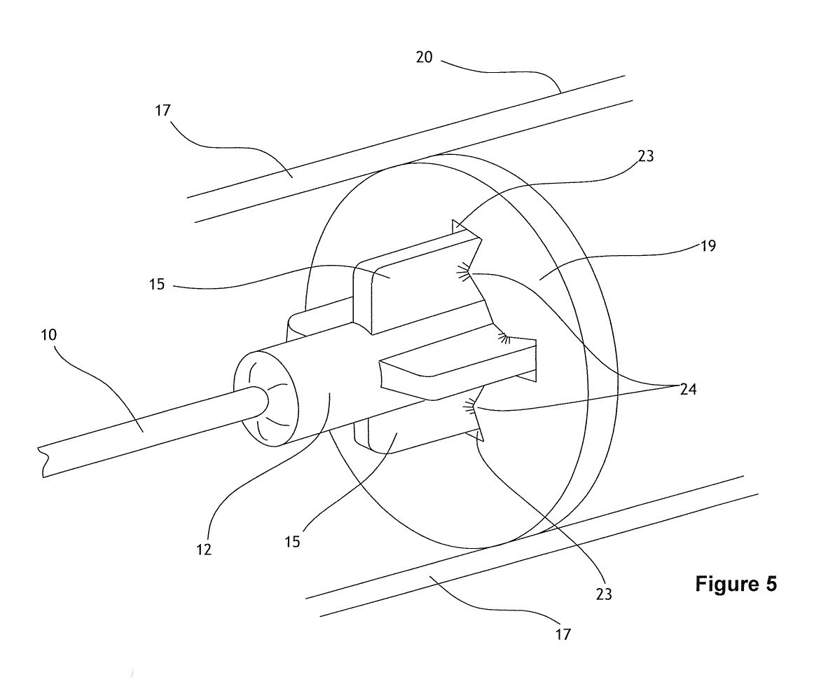

FIG. 4 is an axial section through the needle and hub of FIGS. 1 and 2 but furnished with a mount of this invention;

FIG. 5 is a detail view on an enlarged scale of the important parts of the mount and needle carrier;

FIG. 6 is an end view on a first arrangement of mount and needle carrier;

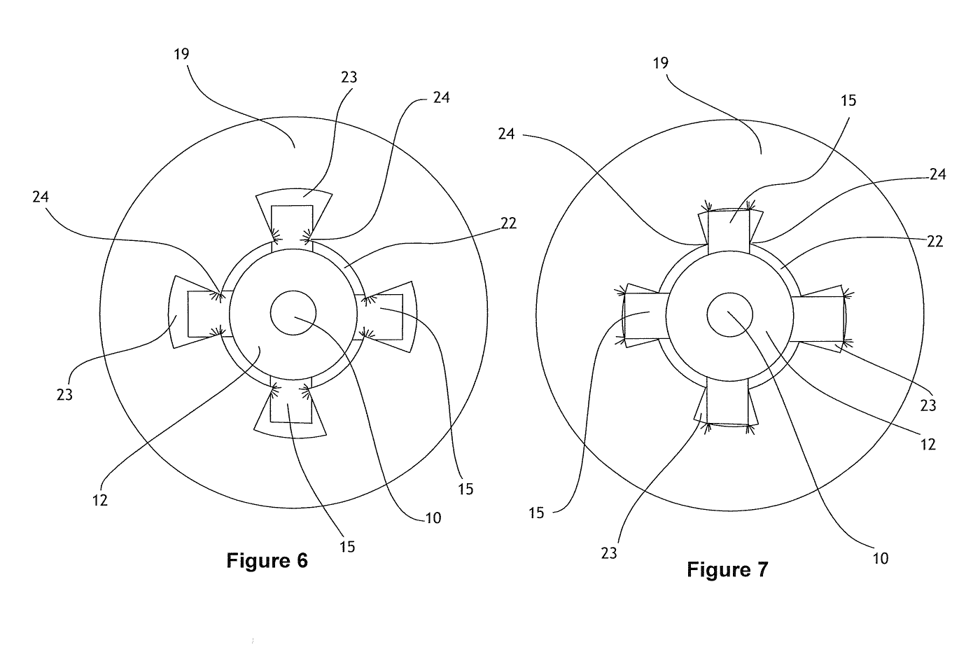

FIG. 7 is an end view on a modified form of the mount and needle carrier arrangement shown in FIG. 6; and

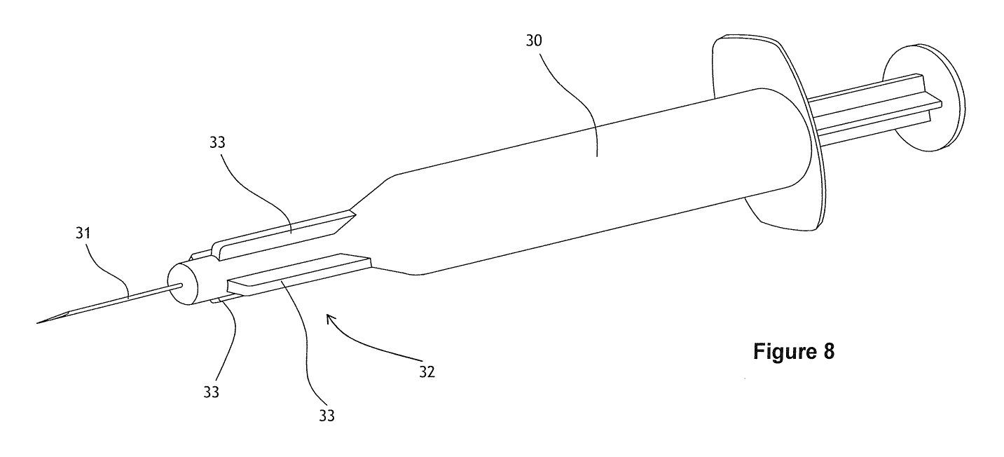

FIG. 8 is a diagrammatic isometric view of a moulded plastics material syringe configured for use with the mount of FIGS. 5 to 7.

Referring initially to FIGS. 1 and 2, there is shown in part a medical needle 10 projecting from a needle hub 11, the end (not shown) of the needle remote from the hub being sharp and used for penetrating a human or animal body, or for other medical purposes. The hub is used to connect to a syringe or other injector (not shown), thereby to mount the needle on that syringe or injector.

The hub has a tubular carrier portion 12 within which the needle is received, the needle being secured therein either means of an adhesive or by being co-moulded with the hub. A conical portion 13 extends from the rearward end of the carrier portion 12 and has an internal conical surface adapted for connection to a Luer-Slip connector formed at the forward end of a syringe body. In the alternative, the conical internal surface could be adapted for use with a Luer-Lok connector as provided on some syringes, to give a more secure connection than a Luer-Slip connector. Surrounding the rearward end of the conical portion is a flange 14 to facilitate handling of the needle and its hub, but with some injectors also forming a part of the connection.

Formed along the external surface of the tubular carrier portion 12 are four ribs 15 arranged mutually at right-angles and extending for the greater part of the length of the carrier portion, the rearward ends of the ribs merging into the external surface of the conical portion 13. Each rib is of substantially rectangular cross-sectional shape and has a rounded forward end which terminates rearwardly of the front of the carrier portion. The hub is typically formed from a plastics material such as polypropylene of an appropriate grade to allow a secure Luer-Slip or Luer-Lok connection. Such a needle and hub arrangement is entirely conventional and is well-known to those skilled in this art.

FIG. 3 shows an alternative form of needle hub. As compared to the hub 11 of FIGS. 1 and 2 having four ribs 15 mutually at right angles, the hub 16 has three ribs 15 equi-spaced circumferentially around the hub—that is to say, with a 120° spacing between the ribs. In all other respects the hub 16 corresponds to the hub 11.

FIGS. 4 to 6 show a mount of this invention in conjunction with a needle and hub as shown in FIGS. 1 and 2. The mount is in the form of a tube 17 having an internal diameter slightly larger than the diameter across opposed ribs 15, such that the tube 17 may slide over those ribs. At its rearward end 18, the sleeve is internally chamfered so as to fit closely against the external surface of the conical portion 13 of the hub. Part way between the ends of the tube, there is provided an internal annular wall 19 profiled to fit around the ribs 15 and the external surface of the tubular carrier portion 12 of the hub 11, as will be described in further detail below.

The tube 17 has a cylindrical outer surface 20 on which is mounted a sliding sleeve 21 adapted to confer protection to the needle 10, that sleeve being slidable rearwardly from a needle shielding position shown in FIG. 3 to a non-shielding position where at least a part of the length of the needle is exposed, for performing a medical procedure. After completion of that procedure, the sleeve may be slid forwardly once more to the position shown in FIG. 3, again to confer protection on the needle. The rearward sliding movement of the sleeve (relative to the needle and hub) may occur by placing the forward end of the sleeve against a patient's skin and pushing the needle forwardly into the body of a patient. The forward sliding movement of the sleeve (again relative to the needle and hub) may occur automatically under the action of a suitable spring (not shown) or by a manual operation performed by a user, after the completion of the medical procedure. In addition, a suitable mechanism (not shown) may be provided to lock the sleeve in its protecting position, when slid there following the performance of the procedure.

The details of the sleeve 21 and its associated arrangements for spring-urging the sleeve forwardly and locking the sleeve in its shielding position after performance of a procedure form no part of the present invention and will not be described in detail here, though appropriate arrangements are well-known and understood by those skilled in the art. Examples of suitable spring and locking arrangements are described and claimed in co-pending international application No PCT/GB2011/050159.

The tube 17 including the annular wall 19 is formed of a plastics material of a harder grade than that from which the needle hub is formed. For example, the tube may be formed of an acetyl plastic of a suitable grade. As best seen in FIGS. 5 and 6, the annular wall 19 has an opening defining a central aperture 22 for fitting closely to the tubular carrier portion 12 of the needle hub 11. Further, the opening has four recesses 23 arranged mutually at right angles with each recess 23 having a dovetail profile in the plane of the wall 19. Each recess 23 thus defines a pair of opposed relatively sharp corners 24 at the junction of the recess with the central aperture. Those corners are spaced radially outwardly from the tubular carrier portion 12 and the spacing between the corners 24 of each recess is slightly less than the cross-sectional width of each rib 15. As each recess is of increasing width in the radially outward direction (in view of the dovetail profile thereof), there will be no contact between the sides of the dovetail profile recess except at the radially innermost end thereof (i.e. in the vicinity of the corners 24). Moreover, the radial depth of each recess 23 is slightly greater than the radial height of each rib whereby there will be no significant contact at the radially outer surface of each rib, with the adjacent part of the annular wall 19.

FIGS. 5 and 6 show the interaction between the corners 24 and the ribs 15. As the annular wall 19 is made of a relatively hard material compared to the needle hub 11, there is an interference fit between the corners 24 and the material of the ribs 15. The corners 24 thus dig into the material of the ribs and so provide a secure connection between the annular wall 19 and the needle hub. In this way, the sleeve 17 is securely attached to the needle hub and any attempt to pull the sleeve 17 off the needle hub will be resisted by the deformed plastics material of the ribs into which the corners 24 of the wall 19 have penetrated. The security of the connection may be enhanced after the sleeve has been located on the ribs for some while, by the material settling into its deformed shape.

In order to allow the required functionality of the corners 24 interacting with the ribs 15, there must be clearance between outer edge of the central aperture 22 extending through the annular wall 19 and the tubular carrier portion 12 of the needle hub. Though in FIG. 6 there is shown a significant clearance between the radially outer faces of the ribs 15 and the surfaces of the wall 19 defining the outermost parts of the recesses 23, in fact that clearance could be reduced almost to nothing, such that the ribs will interact with the annular wall to give guidance thereto as the tube 17 is pushed on to the needle hub 11

FIG. 7 shows an alternative arrangement, where the locking action is provided by the radially outer surfaces of the dovetail recesses 23 interacting with the radially outer faces of each rib 15. In this case, it is not necessary for the radially inner corners 24 of each recess to interact so positively with the ribs 15, as has been described above with reference to FIGS. 5 and 6. Instead, the radially outer surfaces of the recesses 23 may interact with the outer faces in much the same way as with a so-called star-washer fitted to a metal shaft in a manner well known in the engineering art where the star-washer is of a relatively hard grade of metal as compared to the metal shaft with which the star-washer is used.

FIG. 8 shows a plastics material syringe 30 having a needle 31 permanently fitted thereto but where the syringe has been especially configured for use with the mount described above. The nose portion 32 of the syringe emulates the carrier portion of the needle hub shown in FIGS. 1 and 2 and so is moulded to have four ribs 33 arranged mutually at right-angles and extending parallel to the axis of the syringe body, projecting radially outwardly from that part of the forward end of the syringe within which the needle is received. Moulded syringes are well-known but mostly are used with separate needles though increasingly plastics material syringes having needles permanently secured thereto during manufacture are coming on to the market, to replace glass syringes. In order that the needle is adequately secured in such a syringe body, the grade of plastics material from which the syringe body is manufactured must be carefully selected and typically is of a cyclic polyolefin.

It will be appreciated that the profile of the syringe of FIG. 8 in the region of the needle carrier part 32 thereof is very similar to that of a needle hub for the case where the needle and hub are manufactured and separately packaged, for subsequent association with a syringe. As such, the mount described above with reference to FIGS. 4 to 6 or FIG. 7 may be used in the same manner with the syringe of FIG. 8, to allow the mounting of a safety device to the syringe carrying a needle.

The moulds used to form the conventional needle hub shown in FIGS. 1 and 2 or FIG. 3, and also the syringe of FIG. 7, may be relatively simple and not require the use of side actions. This leads to reduced manufacturing costs for these components.

1. A mount for a safety device for use in association with a medical needle projecting from a generally cylindrical carrier including at least two circumferentially-spaced ribs upstanding from and extending substantially parallel to the axis of the carrier, said mount including a wall having a central aperture through which the carrier extends when in use with the wall lying in a substantially radial plane with respect to the carrier, the aperture being profiled so that portions of the wall defining the aperture engage with an interference fit the ribs of the carrier.

2. A mount as claimed in claim 1, wherein the aperture has a generally circular central region adapted to accommodate the carrier and a plurality of recesses extending generally radially away from the central region, each recess being adapted to receive a respective rib upstanding from the carrier.

3. A mount as claimed in claim 2, wherein there is formed a pair of opposed corners at the junction between each recess and the central aperture of said wall, the spacing between the corners being less than the width of a rib on the carrier with which the mount is to co-operate, whereby in use the corners form an interference fit with the associated rib.

4. A mount as claimed in claim 1, wherein each recess has a radially outer surface defined by the material of said wall, whereby in use the outer surfaces of the recesses engage and form an interference fit with the radially outer faces of the associated ribs.

5. A mount as claimed in claim 2, wherein each recess is of dove-tail shape when viewed in the plane of said wall.

6. A mount as claimed in claim 2 and for use with a carrier having two diametrically ribs, wherein the mount has two correspondingly disposed recesses for co-operation with the ribs.

7. A mount as claimed in claim 2 and for use with a carrier having more than two ribs substantially equi-spaced around the carrier, wherein the mount has a plurality of correspondingly disposed recesses for co-operation with the ribs.

8. A mount as claimed in claim 1, wherein the wall is of a relatively hard material with respect to the material of the ribs of a carrier with which the mount is to be used.

9. A mount as claimed in claim 1, wherein at least in the interference fit regions of the wall and carrier, the materials of the respective parts flex to some extent to enhance the connection between the mount and the carrier.

10. A mount as claimed in claim 1, wherein the mount is tubular and said wall extends in a radial plane within the interior of the tubular mount, part-way between the ends thereof.

11. A mount as claimed in claim 1, wherein the outer surface of the mount serves to support a part of a safety device with which the mount is used.

12. A mount as claimed in claim 11, wherein the outer surface of the mount is cylindrical and slidingly supports a part of the safety device with which the mount is used.

13. A mount as claimed in claim 1 in combination with a medical needle projecting from a generally cylindrical carrier including at least two circumferentially-spaced ribs upstanding from and extending parallel to the axis of the carrier, with parts of the wall of the mount forming an interference fit with the ribs of the carrier thereby to hold the mount to the carrier.

14. A mount and carrier combination as claimed in claim 13, wherein the carrier comprises a part of a needle hub which also has a portion adapted for connection to a syringe or other injector.

15. A mount and carrier combination as claimed in claim 13, wherein the carrier comprises a part of a syringe which has a needle secured thereto to project forwardly therefrom, the syringe also having at least two opposed ribs upstanding from and extending parallel to the axis of a carried needle in the region of the mounting of the needle to the syringe.

16. A mount and carrier combination as claimed in claim 13, wherein the carrier has more than two ribs substantially equi-spaced around the carrier, and the mount has a like number of correspondingly disposed recesses for co-operation with the ribs.

17. A mount and carrier combination as claimed in claim 14, wherein the carrier has more than two ribs substantially equi-spaced around the carrier, and the mount has a like number of correspondingly disposed recesses for co-operation with the ribs.

18. A mount and carrier combination as claimed in claim 15, wherein the carrier has more than two ribs substantially equi-spaced around the carrier, and the mount has a like number of correspondingly disposed recesses for co-operation with the ribs.
